(19) United States
(12) Patent Application Publication
(10) Pub. No.: US 2014/0038992 A1
(43) Pub. Date: Feb. 6, 2014

(54) METHODS OF ADMINISTERING RALTEGRAVIR AND RALTEGRAVIR COMPOSITIONS

(71) Applicant: HETERO RESEARCH FOUNDATION, Hyderabad (IN)

(72) Inventors: BANDI PARTHASARADHI REDDY, Hyderabad (IN); MALE SRINIVAS REDDY, Hyderabad (IN); PODILLI KHADGAPATHI, Hyderabad (IN); GOLI KAMALAKAR REDDY, Hyderabad (IN)

(21) Appl. No.: 14/056,254

(22) Filed: Oct. 17, 2013

Related U.S. Application Data
(63) Continuation-in-part of application No. 13/446,114, filed on Apr. 13, 2012.

(30) Foreign Application Priority Data
Apr. 25, 2011 (IN) 1414/CHE/2011
Oct. 22, 2012 (IN) 4386/CHE/2012

(51) Int. Cl. A61K 47/34; A61K 31/513

(52) U.S. Cl. CPC A61K 47/34; A61K 31/513
USPC 514/269

(57) ABSTRACT

The present disclosure is related to amorphous raltegravir and the solid oral dosage forms of amorphous raltegravir that are of lower dosage than the commercially available reference dosage form.

### CROSS-REFERENCE TO RELATED APPLICATIONS

This patent application is a continuation in part of U.S. application Ser. No. 13/446,114 filed on Apr. 13, 2012, which claims priority to Indian patent application number 1414/CHE/2011, filed Apr. 25, 2011; and also claims priority to Indian patent application number 4386/CHE/2012, filed on Oct. 22, 2012, the contents of which are incorporated by reference herein in their entirety.

### FIELD OF THE DISCLOSURE

The present disclosure relates to solid oral dosage forms of raltegravir and methods of administration of raltegravir.

### BACKGROUND

Raltegravir potassium is a human immunodeficiency virus integrase strand transfer inhibitor. The chemical name for raltegravir potassium is N-[(4-Fluorophenyl) methyl]-1,6-dihydro 5-hydroxy-1-methyl-2-[1-methyl-1-[[(5-methyl-1,3,4-oxadiazol-2-yl)carbonyl]amino]ethyl]-6-oxo-4-pyrimidinecarboxamide monopotassium salt, disclosed in U.S. Pat. No. 7,169,780.

Its empirical formula is C₂₀H₂₀FKN₆O₅ and the molecular weight is 482.51, with a structural formula as follows:

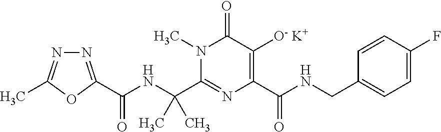

Raltegravir is indicated for the treatment of human immunodeficiency virus (HIV-1) infection. Raltegravir is commercially available from Merck & Co. Inc. as Isentress®. Isentress® is available as a 400 mg film-coated tablet or as 25 mg and 100 mg chewable tablets. In adults, the 400 mg film-coated tablets are administered twice daily.

The common side-effects from administration of Isentress® are trouble sleeping, headache, nausea, tiredness, weakness, stomach pain, dizziness, depression, and suicidal thoughts and actions.

Clinical study data of Isentress® shows that there is high coefficient of variance between the subjects which may lead to drug resistance in patients.

What is needed are alternative dosage forms and methods of administering raltegravir potassium.

### SUMMARY

In one aspect, the present disclosure relates to solid oral dosage forms of raltegravir and one or more pharmaceutically acceptable excipients.

In one aspect, described herein is a pharmaceutical solid oral dosage form comprising (a) 300 mg of raltegravir or an equivalent amount of amorphous raltegravir in the form of a pharmaceutically acceptable salt thereof; (b) a polymer; and (c) a pharmaceutically acceptable excipient. In one embodiment, the pharmaceutically acceptable excipient comprises a polymer such as a polyethylene oxide or a polymethacrylate polymer.

In another aspect, a method of reducing inter subject variability during administration of raltegravir to a human subject comprises administering to the human subject a solid oral dosage form comprising 300 mg of amorphous raltegravir or an equivalent amount of a pharmaceutically acceptable salt thereof.

In yet another aspect, a method of reducing adverse effects upon administration of raltegravir comprises administering to the human subject a solid oral dosage form comprising 300 mg of amorphous raltegravir or an equivalent amount of a pharmaceutically acceptable salt thereof.

### DETAILED DESCRIPTION

Described herein are solid oral dosage forms containing raltegravir and a pharmaceutically acceptable excipient. The solid oral dosage forms contain 300 mg of raltegravir or an equivalent amount of raltegravir in the form of a pharmaceutically acceptable salt thereof, which is substantially lower than the 400 mg of raltegravir in the currently marketed Isentress® tablet. In certain embodiments, a 300 mg solid oral dosage form as described herein is bioequivalent to Isentress® tablets containing 400 mg of raltegravir.

In one embodiment, raltegravir is in the form of raltegravir potassium. In another embodiment, the raltegravir such as raltegravir potassium is in amorphous form.

The raltegravir is present in the solid oral dosage form in an amount of 10% to 60% by weight, based on the total weight of the dosage form.

A “pharmaceutical composition” comprises an active pharmaceutical ingredient and a pharmaceutically acceptable excipient. The term “pharmaceutically acceptable excipient” includes a pharmaceutically acceptable material, polymer or vehicle, suitable for administering an active pharmaceutical ingredient. Each excipient should be “acceptable” in the sense of being compatible with the other ingredients of the formulation and not injurious to the patient.

A solid oral dosage form is a pharmaceutical composition suitable for administration in a single dose, such as a tablet or capsule or granules.

In one embodiment, the 300 mg raltegravir solid oral dosage form comprises a polymer. Exemplary polymers include polyethylene oxide; polymethacrylate polymers such as poly(ethyl acrylate, methyl methacrylate, trimethylammonioethyl methacrylate chloride), poly(ethyl acrylate, methyl methacrylate), a methacrylic acid-methyl methacrylate co-polymer, a methacrylic acid-ethyl acrylate co-polymer, poly(methyl acrylate, methyl methacrylate, methacrylic acid); and hydroxyalkyl celluloses such as hydroxypropyl cellulose, hydroxylethyl cellulose, hydroxyalkyl alkylcellulose, hydroxypropyl methylcellulose, hydroxyethylmethyl cellulose; and combinations thereof. In a specific aspect, the polymer is a polyethylene oxide.

It is noted that polyethylene oxide is different from polyethylene glycol. Polyethylene oxide is a high molecular weight non ionic homopolymer of ethylene oxide and having the molecular formula (CH₂CH₂O)ₙ. In comparison, polyethylene glycol is a low molecular weight addition polymer of ethylene oxide and water, and having the molecular formula is H(OCH₂CH₂)ₙOH, where n represents the average number of oxyethylene groups.

The polymer is present in the oral dosage form in an amount of 0.5% to 20% by weight, specifically 1% to 10% by weight, based on the total weight of the dosage form.

The 300 mg raltegravir potassium formulation optionally comprises a solubilizing agent. Exemplary solubilizing agents include polyoxyethylene-polyoxypropylene block copolymers (also known as poloxamers), polysorbate, sodium lauryl sulphate, soluplus (a graft copolymer comprised of polyethylene glycol, polyvinylcaprolactam and polyvinylacetate), polyethylene glycols, sodium stearyl sulfate, sodium oleyl sulfate, sodium cetyl sulfate, sodium dodecylbenzene sulfonate, dialkyl sodium sulfosuccinates, fatty acid macrogolglycerides—an exemplary fatty acid macrogolglyceride is stearoyl macrogolglyceride, such as GELUCIRE® 50/13 (available from Gattefosse, Paramus, N.J.) which is a mixture of mono-, di- and triglycerides and mono- and di-fatty acid esters of polyethylene glycol and the like, and combinations thereof.

The formulations further comprise additional excipients such as diluents, binders, disintegrants, glidants, lubricants, and optionally flavoring agents and others. The diluent, for example, is present in an amount 0 to 85% by weight of the tablet core. The total amount of additional excipients is 0 to 89.5% by weight, based on the total weight of the dosage form.

Diluents increase the bulk of a solid pharmaceutical composition. Exemplary diluents for solid compositions include, but are not limited to, microcrystalline cellulose, lactose, dibasic calcium phosphate, microfine cellulose, starch, pregelatinized starch, calcium carbonate, calcium sulfate, sugar, dextrates, dextrin, dextrose, tribasic calcium phosphate, kaolin, magnesium carbonate, magnesium oxide, maltodextrin, mannitol, polymethacrylates, potassium chloride, powdered cellulose, sodium chloride, sorbitol, talc, and combinations thereof.

Solid pharmaceutical compositions that are compacted into a dosage form, such as a tablet, may include excipients whose functions include helping to bind the active ingredient and other excipients together after compression. Exemplary binders for solid pharmaceutical compositions include, but are not limited to, acacia, alginic acid, carbomer, carboxymethylcellulose sodium, dextrin, ethyl cellulose, gelatin, guar gum, hydrogenated vegetable oil, hydroxyethyl cellulose, hydroxypropyl cellulose, hydroxypropyl methyl cellulose, liquid glucose, magnesium aluminum silicate, maltodextrin, methylcellulose, polymethacrylates, povidone, pregelatinized starch, sodium alginate, starch, and combinations thereof.

Disintegrants increase the dissolution rate of a compacted solid pharmaceutical composition in the patient's stomach, for example. Exemplary disintegrants include, but are not limited to, microcrystalline cellulose, alginic acid, carboxymethylcellulose calcium, carboxymethylcellulose sodium, colloidal silicon dioxide, croscarmellose sodium, crospovidone, guar gum, magnesium aluminum silicate, methyl cellulose, polacrilin potassium, powdered cellulose, pregelatinized starch, sodium alginate, sodium starch glycolate and starch.

Glidants can be added to improve the flowability of a non-compacted solid composition and to improve the accuracy of dosing. Exemplary glidants include, but are not limited to, colloidal silicon dioxide, magnesium trisilicate, powdered cellulose, starch, talc and tribasic calcium phosphate.

Compaction of a powdered composition into a tablet, for example, subjects the composition to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Exemplary lubricants include, but are not limited to, magnesium stearate, sodium stearyl fumarate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, stearic acid, talc and zinc stearate.

Solid compositions include powders, granulates, aggregates and compacted compositions. The dosages may be conveniently presented in unit dosage form and prepared by methods well-known in the pharmaceutical arts.

Dosage forms include solid dosage forms like tablets, powders, granules, capsules, suppositories, sachets, troches and lozenges, as well as liquid syrups, suspensions and elixirs.

The active ingredient and excipients may be formulated into compositions and dosage forms according to methods known in the art.

A tableting composition may be prepared by dry blending. For example, the blended composition of the actives and excipients may be compacted into a slug or a sheet and then comminuted into compacted granules. The compacted granules may subsequently be compressed into a tablet.

Compositions for tableting or capsule filling may be prepared by wet granulation. In wet granulation, active ingredient and some or all of the excipients are blended and then further mixed in the presence of a liquid, that causes the powders to clump into granules. The granulate is screened and/or milled, dried and then screened and/or milled to the desired particle size. The granulate may then be tableted/filled, or other excipients may be added prior to tableting/filling, such as a glidant and/or a lubricant.

As an alternative to granulation, a blended composition may be compressed directly into a compacted dosage form using direct compression techniques. Excipients that are particularly well suited for direct compression tableting include microcrystalline cellulose, spray dried lactose, dicalcium phosphate dihydrate and colloidal silica.

A process for preparing pharmaceutical compositions comprising 300 mg of amorphous raltegravir or an equivalent amount of amorphous raltegravir in the form of a pharmaceutically acceptable salt thereof, a polymer, and one or more pharmaceutically acceptable excipients, comprises using dry granulation, wet granulation, spray granulation, direct compression or extrusion and spheronization.

Pharmaceutical compositions are optionally “coated”. The coating can be a suitable coating, such as, a functional or a non-functional coating, or multiple functional coatings. A “functional coating” is a coating that modifies the release properties of the total formulation, for example, a sustained-release coating. A “non-functional coating” is a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition. In one embodiment, a coating is a substantially uniform coating.

The formulations described herein may be coated with a functional or non-functional coating. The coating may comprise about 0 wt % to about 10 wt % of the composition. The coating material may include a polymer, preferably a film-forming polymer, for example, methyl cellulose, ethyl cellulose, hydroxypropyl cellulose, hydroxypropyl methyl cellulose, hydroxybutyl methyl cellulose, cellulose acetate, cellulose propionate, cellulose acetate propionate, cellulose acetate butyrate, cellulose acetate phthalate, carboxymethyl cellulose, cellulose triacetate, cellulose sulphate sodium salt, poly(methyl methacrylate), poly(ethyl methacrylate), poly(butyl methacrylate), poly(isobutyl methacrylate), poly(hexyl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, (poly propylene), poly(ethylene glycol poly(ethylene oxide), poly(ethylene terephthalate), poly(vinyl alcohol), poly(vinyl isobutyl ether), poly(vinyl acetate), poly(vinyl chloride), polyvinyl pyrrolidone, and combinations comprising one or more of the foregoing polymers.

The inclusion of an effective amount of a plasticizer in the coating composition may improve the physical properties of the film.

As used herein, a polymorph is a crystalline form of an active pharmaceutical ingredient having a distinguishable crystalline form, and includes crystalline polymorphs, amorphous forms, as well as solvate and hydrate forms, which are often referred to as pseudopolymorphs. Solvates are crystalline forms containing a stoichiometric or nonstoichiometric amount of solvent. A hydrate is a solvate wherein the solvent is water.

Amorphous forms are disordered forms that do not have a distinguishable crystalline lattice. Amorphous materials typically do not have sharp, well-defined reflections in their x-ray diffraction patterns, but rather a broad peak spanning a range of two-theta angles.

Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning calorimetry (DSC) and Infrared spectrometry (IR).

Active pharmaceutical ingredient can be in a free acid or free base form, a salt, a hydrate, or an anhydrate, for example.

An active pharmaceutical ingredient can be used as a pharmaceutically acceptable salt. As used herein a “pharmaceutically acceptable salt” is a salt of an acidic or basic group that is non-toxic. Exemplary acids used to form pharmaceutically acceptable salts include containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, mesylate, saccharate, formate, benzoate, glutamate, methanesulfonate, ethanesulfonate, benzenesulfonate, p-toluenesulfonate and pamoate salts. Exemplary bases include sodium hydroxide, sodium carbonate, potassium hydroxide, aluminum hydroxide, calcium hydroxide, ethanolamine, diethanolamine, triethanolamine, tromethamine, N-methylglucamine and the like.

Pharmaceutical dosage forms can vary in their release properties. By “immediate release composition” is meant a dosage form that is formulated to release substantially all the active pharmaceutical ingredient on administration with no enhanced, delayed or extended release effect. In immediate-release greater than or equal to about 75% of the active agent is released within two hours of administration, specifically within one hour of administration.

Both “extended-release” and “delayed release” are modified or controlled forms of release. In extended-release, the drug is released over a period of time, such as, for example 4, 6, 10, 12, 15, 18, 21, or 24 hours, or more. Suitable materials for release control include EUDRAGIT® RL (copolymers of acrylic and methacrylic acid esters), EUDRAGIT® RS (copolymers of acrylic and methacrylic acid esters), cellulose derivatives such as ethylcellulose aqueous dispersions (AQUACOAT®, SURELEASE®), hydroxyethyl cellulose, hydroxypropyl cellulose, hydroxypropyl methylcellulose, polyvinyl pyrrolidone, polyvinylpyrrolidone/vinyl acetate copolymer, and the like.

“Delayed release” is release delayed for a period of time after administration and can be accomplished, for example, by applying a coating of enteric materials. “Enteric materials” are polymers that are substantially insoluble in the acidic environment of the stomach, but are predominantly soluble in intestinal fluids at various specific pHs.

In certain cases, dosage forms can have both immediate-release and controlled-release properties, such as a combination of immediate-release and controlled-release pellets.

The dissolution properties of a dosage form can be tested by methods known in the art. An exemplary condition is a USP type 2 (paddle) apparatus at 100 rpm in 900 ml of water. Alternatively, a basket method may be employed.

In certain embodiments, the formulations described herein are bioequivalent to the reference dosage form, in this case Isentress®. The term “bioequivalent” means the absence of a significant difference in the rate and extent to which the active pharmaceutical ingredient or active moiety in pharmaceutical equivalents or pharmaceutical alternatives becomes available at the site of drug action when administered at the same molar dose under similar conditions as Isentress® in an appropriately designed study. Isentress® is a tablet containing hydroxypropylmethylcellulose, poloxamer, as well as microcrystalline cellulose, lactose monohydrate, calcium phosphate, sodium stearyl fumarate, and magnesium stearate. The reference dosage form also comprises a film coating.

In one embodiment, a method of treating HIV-1 infection in a patient in need thereof comprises orally administering a 300 mg of raltegravir twice daily over a treatment period.

The compositions described herein are particularly useful for treating patients with HIV-1 infection.

Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must be between 0.70 to 1.43.

“Pharmacokinetic parameters” are parameters which describe the in vivo characteristics of the active agent over time, including for example the in vivo dissolution characteristics and plasma concentration of the active agent. By “Cₘₐₓ” is meant the measured concentration of the active agent in the plasma at the point of maximum concentration. By “C₂₄” is meant the concentration of the active agent in the plasma at about 24 hours. The term “Tₘₐₓ” refers to the time at which the concentration of the active agent in the plasma is the highest. “AUC” is the area under the curve of a graph of the concentration of the active agent (typically plasma concentration) vs. time, measured from one time to another.

The current label for Isentress® 400 mg tablets indicates that the tablets exhibit considerable inter-patient variability. “Considerable variability was observed in the pharmacokinetics of raltegravir. For observed C12 hr in Protocols 018 and 019, the coefficient of variation (CV) for inter-subject variability=212% and the CV for intra-subject variability=122%”. The existence of high inter subject variability of Isentress® 400 mg tablets was further confirmed from the pilot studies shown herein (Refer to Table-1).

The bioavailability of Isentress® 400 mg tablets was reported to be 68% as per the literature.

Described in the Examples herein are tablet compositions comprising 400 mg of amorphous raltegravir with one or more pharmaceutically acceptable excipients (herein referred to as the test product). The test product matched the dissolution profile of Isentress® 400 mg tablets (herein referred to as the reference product). Based on this result, in vivo studies were conducted.

Based on in-vivo study results, the test product showed higher bioavailability, i.e., about 90% bioavailability, and reduced inter-subject variability compared to the reference product which had high inter-subject variability (Refer to Table-2).

From the in vivo studies, it was concluded that the test product formulated with amorphous raltegravir 400 mg had a higher bioavailability than Isentress® 400 mg tablets (Refer Table-3).

As the test product had a greater bioavailability than Isentress® 400 mg tablets, tablet compositions of amorphous raltegravir with a reduced dose were developed.

Accordingly, the tablet compositions of amorphous raltegravir with a reduced dose of 300 mg were formulated and subjected to in-vitro dissolution studies. After matching the in-vitro dissolution profiles, the test product was subjected to pilot studies.

Based on the pilot study results (Refer to Table-4 and Table-5), it was concluded that a dosage of 300 mg of amorphous raltegravir (T) may be sufficient to provide bioequivalence to Isentress® 400 mg tablets (R) comprising substantially crystalline raltegravir. Further, the 300 mg tablets provide fewer side effects and are more economical.

Moreover, it was found that the dosage of 300 mg of amorphous raltegravir (T) showed fixed absorption with the required T/R Ratio, i.e., 80% to 125% in all the subjects. Without being held to theory, the improved absorption of amorphous raltegravir could be due to high purity nature of amorphous Raltegravir. This in turn reduces the side effects due to the reduced dosage strength.

As predicted, the pharmacokinetic profiles of a lesser dose (300 mg) of amorphous raltegravir are similar to Isentress® 400 mg and product is bioequivalent (as per FDA guidelines*).

[*: As per U.S. FDA guidelines, two products are bioequivalent if the 90% Confidence Intervals (CI) for AUC and Cₘₐₓ are between 0.80 to 1.25 (Tₘₐₓ measurements are not relevant to bioequivalence for regulatory purposes.]

Based on the studies herein, a pharmaceutical composition containing 300 mg of amorphous raltegravir was found to have the following advantages:
Bioavailability increased from 65 to 90%
Reduced side effects since there is a reduced dose
low variation in pharmacokinetic profile which ultimately leads to uniform therapeutic effect with reduced drug resistance
Reduced drug load disposition
Economical
Medicinally therapeutically equivalent

An oral dosage form as described herein is characterized by its pharmacokinetics, such as Cmax(average concentration of active chemical in the bloodstream after oral ingestion, preferably in the fasted state). In a specific embodiment, the oral dosage form provides only one peak concentration when blood concentrations are plotted against time.

In one embodiment, a method of reducing inter subject variability during administration of raltegravir to a human subject comprises administering to the human subject an oral dosage form comprising 300 mg of amorphous raltegravir or an equivalent amount of a pharmaceutically acceptable salt thereof. In one embodiment the inter-subject coefficient of variability for Cmaxof a 300 mg raltegravir dosage form as described herein is less than 100%, less than 90%, less than 80%, less than 70% or less than 60%. In another embodiment, the oral dosage form provides only one peak concentration when blood concentrations are plotted against time after administration.

In another embodiment, a method of reducing adverse effects upon administration of raltegravir comprises administering to the human subject an oral dosage form comprising 300 mg of amorphous raltegravir or an equivalent amount of a pharmaceutically acceptable salt thereof.

In one embodiment, a method of treating HIV-1 infection in a patient in need thereof comprises orally administering a 300 mg raltegravir oral dosage form as described herein twice per day, to give a daily dose of 600 mg. In another embodiment, administration is over a treatment period such as, for example, 24 weeks or longer. In certain embodiments, the subject is an adult, a child 12 years of age or older, or a child 6 to less than 12 years of age. In certain embodiments, the dosage forms are administered with and without food.

TABLE 1
Pilot study results of Isentress ® 400 mg tablets

| | Cₘₐₓ | | | | |
|---|---|---|---|---|---|
| Subject | Study I | Study II | Study III | Study IV | Study V |
| 1 | 5674.05 | 6129.65 | 4262.99 | 7281.96 | 8207.65 |
| 2 | 1984.44 | 17112.32 | 599.04 | 992.13 | 2981.06 |
| 3 | 4276.37 | 5330.11 | 5092.69 | 7268.83 | 8587.65 |
| 4 | 6384.64 | 7903.41 | 8314.97 | 5462.36 | 14182.94 |
| 5 | 2724.73 | 1064.98 | 940.16 | 1443.92 | 5137.76 |
| 6 | 3481.62 | 4103.62 | 350.9 | 1384.17 | 1453.52 |
| 7 | 1213.9 | 9051.42 | 5123.25 | 8653.91 | 12586.36 |
| 8 | 1558.03 | 8722.77 | 5331.5 | 4424.69 | 4990.72 |
| 9 | 6066.62 | 8054.69 | 7845.94 | 3775.96 | 1121.76 |
| 10 | 4096.02 | 7419.88 | 9158.75 | 7827.64 | 170.57 |
| 11 | 5016.62 | 4768.27 | 3313.3 | 9410.8 | 398.47 |
| 12 | 8257.78 | 2652.3 | 8243.37 | 2539 | 2725.77 |
| 13 | 2801.89 | — | 9990.55 | 4810.57 | 450.94 |
| 14 | 4887.71 | — | 11038.78 | 8497.14 | 2703.62 |
| MEAN | 4173.173 | 6859.452 | 5686.157 | 4964.25 | 4692.771 |
| SD | 2028.534 | 4071.472 | 3537.393 | 3068.925 | 4566.335 |
| MIN | 1213.9 | 1064.98 | 350.9 | 690.67 | 170.57 |
| MAX | 8257.78 | 17112.32 | 11038.78 | 9410.8 | 14182.94 |
| CV % | 48.6 | 59.4 | 62.2 | 61.8 | 97.3 |

From the Table-1 it is observed that for Isentress® there is high coefficient of variance between the subjects which leads to drug resistance within the patients.

TABLE 2
Pilot study results of Test Raltegravir 400 mg Tablets

| | Cₘₐₓ | | | | |
|---|---|---|---|---|---|
| Subject | Study I | Study II | Study III | Study IV | Study V |
| 1 | 4363.95 | 8520.7 | 10032.52 | 1023.37 | 4828.26 |
| 2 | 4506.82 | 15470.8 | 6676.89 | 5235.94 | 3160.18 |
| 3 | 6049.81 | 5593.97 | 8182.19 | 5075.97 | 5965.18 |
| 4 | 4953.47 | 7009.71 | 7903.51 | 15761.39 | 8364.86 |
| 5 | 2266.94 | 5204.94 | 5812.84 | 6060.98 | 10481.01 |
| 6 | 3455.06 | 10539.03 | 11085.75 | 5408.22 | 8091.46 |
| 7 | 1943.37 | 8163.68 | 10748.79 | 4418.8 | 4749.96 |
| 8 | 3925.04 | 10235.57 | 8590.92 | 9764 | 14423.17 |
| 9 | 5022.43 | 9450.49 | 9313.4 | 11742.99 | 3600.22 |
| 10 | 4954.45 | 4652.68 | 16520.62 | 4411.86 | 5559.03 |
| 11 | 5133.17 | 7113.51 | 9006.61 | 11622.09 | 8697.69 |
| 12 | 10169.67 | 4853.9 | 9772.25 | 5976.6 | 8759.61 |
| 13 | 3044.13 | — | 11541.78 | 6433.99 | 2109.6 |
| 14 | 3979.86 | — | 13427.92 | 8267.9 | 5745.41 |
| MEAN | 4554.86 | 8067.41 | 9901.43 | 7050.21 | 6752.54 |
| SD | 1979.60 | 3105.34 | 2745.91 | 3746.58 | 3283 |
| MIN | 1943.37 | 4652.68 | 5812.84 | 1023.27 | 2109.6 |
| MAX | 10169.67 | 15470.8 | 16520.62 | 15761.39 | 14423.17 |
| CV % | 43.5 | 38.5 | 27.7 | 53.1 | 48.6 |

TABLE 3

| | Cₘₐₓ | | |
|---|---|---|---|
| | Test (T) | Reference (R) | |
| | Raltegravir 400 mg | Isentress ® 400 mg | T/R ratio |
| Parameter | tablets | tablets | (Fasting) |
| Study III | 9901.43 | 5686.157 | 174.13 |
| Study IV | 7050.21 | 4964.25 | 142.01 |
| Study V | 6752.54 | 4692.771 | 143.89 |

From the Table-2 and Table-3, it is observed that there is high bioavailability with low variation for raltegravir 400 mg tablets of the present disclosure.

TABLE 4
Pilot Fasting B.E study results of amorphous Raltegravir 300 mg tablets
(T) (Example 1) vs. Isentress ® 400 mg tablets (R)

| | GeoLSM | | |
|---|---|---|---|
| | Test (T) | Reference (R) | |
| | Raltegravir 300 mg | Isentress ® 400 mg | T/R ratio |
| Parameter | tablets | tablets | (Fasting) |
| Cmax | 3793.06 | 3120.42 | 121.55 |
| AUC 0-t | 11178.47 | 10378.01 | 107.71 |
| AUC 0-∞ | 11341.26 | 10932.23 | 103.74 |

TABLE 5
Pilot Fed B.E study results of amorphous Raltegravir 300 mg
tablets (T) (Example 1) vs. Isentress 400 mg tablets (R)

| | GeoLSM | | |
|---|---|---|---|
| | Test (T) | Reference (R) | |
| | Raltegravir 300 mg | Isentress ® 400 mg | T/R ratio |
| Parameter | tablets | tablets | (Fed) |
| Cmax | 3661.13 | 3443.14 | 106.33 |
| AUC 0-t | 9594.72 | 9917.64 | 96.74 |
| AUC 0-∞ | 9720.65 | 10098.14 | 96.26 |

From the Table-4 and Table-5, it is observed that amorphous Raltegravir 300 mg tablets are bioequivalent to Isentress 400 mg tablets both in fasting and fed conditions basing on the T/R ratios.

### Biostudy Details

In this study 12+2 (stand-by) volunteers were enrolled at least 12 hours prior to drug administration. The study has two periods and two treatments, meaning that all the subjects received either Test (T) or Reference (R) drug. The periods were separated by a washout period of at least 07 days to ensure that the drug taken during one period is no longer in the subject's system when the next period begins.

A total of 27 blood samples were collected from each subject. The venous blood samples (5.0 mL each) were withdrawn at pre-dose within one hrs and 5 mL at 00.00, 00.33, 00.67, 01.00, 01.33, 01.67, 02.00, 02.25, 02.50, 02.75, 03.00, 03.25, 03.50, 03.75, 04.00, 04.50, 05.00, 05.50, 06.00, 07.00, 08.00, 09.00, 10.00, 12.00, 16.00, 24.00 and 36.00 hours post dose blood samples were collected after drug administration in each period.

In each of the study period, single dose of one tablet of Test product [Raltegravir 300 mg tablets] or co-administration of single tablet of individual Reference product [Isentress®] containing Raltegravir 400 mg tablets were administered to each subject with 240±2 mL of water at room temperature as per the randomization schedule. After a supervised over-night fasting (at least 10 hours) dosing will be done. A standardized food was provided to all the subjects before the night of check in and at 04.00, 09.00 and 13.00 hours (lunch, snacks and dinner) post dose. A window period of 10 minutes was taken into consideration for the administration of food during housing and any deviation is noted as late food administration and is recorded in the respective CRF. Drinking water was restricted for one hour pre-dose to one hour post-dose.

Pharmacokinetic analysis was performed using WinNonlin®-Professional Version 5.3 or higher for Windows at sponsor site. Data from 12 subjects who have completed both the periods were analyzed.

AUC₀₋ₜ: The area under the plasma concentration versus time curve, from time zero to the last measurable concentration, as calculated by the linear trapezoidal method.

AUC_{0-inf}: The area under the plasma concentration versus time curve, from time zero to infinity. AUC_{0-inf} is calculated as the sum of AUC₀₋ₜ plus the ratio of the last measurable plasma concentration to the elimination rate constant.

Cₘₐₓ: Maximum measured plasma concentration over the time span specified.

Tₘₐₓ: Time of the maximum measured plasma concentration. If the maximum value occurs at more than 1 time point, Tₘₐₓ is defined as the first time point with this value.

Kₑₗ: Apparent first-order terminal elimination rate constant calculated from a semi-log plot of the plasma concentration versus time curve. The parameter will be calculated by linear least-square regression analysis using the maximum number of points in the terminal log-linear phase (e.g. three or more non-zero plasma concentrations).

T_{1/2}: The apparent first-order terminal elimination half-life will be calculated as 0.693/Kₑₗ.

No value of Kₑₗ, AUC_{0-inf} or T_{1/2} will be reported for cases that do not exhibit a terminal log-linear phase in the concentration versus time profile.

If pre-dose concentration is found to be less than or equal to 5% of mean Cₘₐₓ, the value was considered as such for calculation. If pre-dose concentration is found to be more than 5% of mean Cₘₐₓ, the respective subject was eliminated from the analysis.

The actual time of blood sample collection was used during pharmacokinetic calculations for blood samples collected later to window period (2 minutes for in-house samples) and earlier or later to window period (2 hours for ambulatory samples).

Summary Statistics: Arithmetic means, standard deviations range (maximum and minimum) and coefficients of variation are calculated for plasma concentrations and for all pharmacokinetic parameters. Additionally, geometric means and percentage coefficient of geometric means are also calculated for Cₘₐₓ, AUC₀₋ₜ and AUC_{0-∞}.

Analysis of Variance (ANOVA): The log-transformed pharmacokinetic parameters (Cₘₐₓ, AUC₀₋ₜ and AUC_{0-∞}) were analyzed using a mixed effects ANOVA model using Type III sum of squares, with the main effects of sequence, period and formulations as fixed effects and subjects nested within sequence as random effect. A separate ANOVA model is used to analyze each of the parameters. The sequence effect is tested at the 0.10 level of significance using the subjects nested within sequence mean square as the error term. All other main effects are tested at the 0.05 level of significance against the residual error (mean square error) from the ANOVA model as the error term. Each analysis of variance will include calculation of least-squares means, the difference between the adjusted formulation means and the standard error associated with the difference. The above analysis is performed using suitable software at sponsor's site.

Bioequivalence criteria: Based on the statistical results of 90% confidence intervals of the ratios of the means (Test/Reference) for ln-transformed pharmacokinetic parameters Cₘₐₓ, AUC₀₋ₜ and AUC_{0-∞}, it was concluded that the test product is bioequivalent to the reference product.

Bioequivalence was concluded, as the Test to Reference (T/R) ratios and the 90% confidence interval for the ratios of means fall within the acceptance range of 80.00-125.00% for ln-transformed data for the pharmacokinetic parameters, Cₘₐₓ, AUC₀₋ₜ and AUC_{0-∞}.

The following examples further illustrate the invention and do not limit the scope of the invention.

### Example 1-2

Tablet compositions of Raltegravir 300 mg:

| | Example -1 | Example - 2 |
|---|---|---|
| Ingredients | mg/tablet | mg/tablet |
| Intra-granular ingredients | | |
| Raltegravir potassium | 325.80 | 325.80 |
| Microcrystalline cellulose | 214.75 | 211.49 |
| Lactose monohydrate | 9.75 | 9.75 |
| Polyethylene oxide* | 22.84 | 26.10 |
| Magnesium stearate | 5.25 | 5.25 |
| Sodium stearyl fumarate | 3.00 | 3.00 |
| Extra-granular ingredients | | |
| Microcrystalline cellulose | 64.58 | 64.58 |
| Magnesium stearate | 6.53 | 6.53 |
| Core tablet weight | 652.50 | 652.50 |
| Coating | | |
| Opadry ® II Pink | 19.58 | 19.58 |
| Purified water | q.s. | q.s. |
| Total tablet weight | 672.08 | 672.08 |

*Polyox WSR 301

### Brief Manufacturing Process:

(i) Intra-granular ingredients were sifted and blended together,
(ii) the blend of step (i) was slugged/compacted and the resulted slugs/compacts were milled using multimill or cone mill,
(iii) milled granules of step (ii) were sifted through mesh 30 # sieve,
(iv) extra-granular ingredients (if any) were sifted together through mesh 40 # sieve,
(v) extra-granular magnesium stearate was sifted through mesh 60 # sieve,
(vi) materials of step (iii), (iv) and (v) were blended together and compressed into tablets,
(vii) compressed tablets of step (vi) were optionally coated with Opadry® II Pink.

### Study on Dissolution:

Dissolution test was performed for tablets prepared as per the Example 1 and Example 2, using USP apparatus II, 100 rpm, in 900 ml of water (deaerated).

| | Time in minutes | | | | | |
|---|---|---|---|---|---|---|
| | 15 min | 30 min | 45 min | 60 min | 90 min | 120 min |
| Example-1 | 40 | 61 | 74 | 84 | 95 | 98 |
| Dissolution (%) | | | | | | |
| Example-2 | 38 | 59 | 76 | 83 | 94 | 98 |
| Dissolution (%) | | | | | | |

### Example 3-4

Tablet compositions of Raltegravir 300 mg:

| | Example -3 | Example - 4 |
|---|---|---|
| Ingredients | mg/tablet | mg/tablet |
| Intra-granular ingredients | | |
| Raltegravir potassium | 325.80 | 325.80 |
| Microcrystalline cellulose | 183.30 | 196.35 |
| Lactose monohydrate | 39.15 | 39.15 |
| Dibasic calcium phosphate | 55.38 | 55.38 |
| Hydroxypropyl methylcellulose | 19.54 | 19.54 |
| Poloxamer | 13.05 | — |
| Magnesium stearate | 3.02 | 3.02 |
| Sodium stearyl fumarate | 6.51 | 6.51 |
| Extra-granular ingredients | | |
| Magnesium stearate | 6.75 | 6.75 |
| Core tablet weight | 652.50 | 652.50 |
| Coating | | |
| Opadry ® II Pink | 19.58 | 19.58 |
| Purified water | q.s. | q.s. |
| Total tablet weight | 672.08 | 672.08 |

### Brief Manufacturing Process:

Same as that of Example-1.

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the specification.

We claim:

1. A pharmaceutical solid oral dosage form comprising:
(a) 300 mg of raltegravir or an equivalent amount of amorphous raltegravir in the form of a pharmaceutically acceptable salt thereof;
(b) a polymer; and
(c) a pharmaceutically acceptable excipient.

2. The pharmaceutical solid oral dosage form of claim 1, wherein the raltegravir salt is raltegravir potassium.

3. The pharmaceutical solid oral dosage form of claim 1, wherein the polymer is a polyethylene oxide or a polymethacrylate.

4. The pharmaceutical solid oral dosage form of claim 1, wherein the polymer is a polyethylene oxide.

5. The pharmaceutical solid oral dosage form of claim 4, wherein the 300 mg raltegravir dosage form is bioequivalent in Cmax, AUC₀₋ₜ and AUC_{0-∞} to a 400 mg raltegravir dosage form containing crystalline raltegravir.

6. The pharmaceutical solid oral dosage form according to claim 1, which is a tablet, granules or a capsule.

7. A method of reducing inter subject variability during administration of raltegravir to a human subject, comprising
administering to the human subject a solid oral dosage form comprising 300 mg of amorphous raltegravir or an equivalent amount of a pharmaceutically acceptable salt thereof.

8. The method of claim 7, wherein the subject is an adult, a child 12 years of age or older, or a child 6 to less than 12 years of age.

9. The method of claim 7, wherein the inter subject coefficient of variability for Cmaxis less than 60%.

10. The method of claim 7, wherein the solid oral dosage form provides only one peak concentration when blood concentrations are plotted against time after administration.

11. The method of claim 7, wherein administering is twice per day

12. The method of claim 7, wherein the solid oral dosage form comprises a polymer selected from polyethylene oxide and polymethacrylate polymers.

13. The method of claim 12, wherein the 300 mg raltegravir dosage form is bioequivalent in Cmax, AUC₀₋ₜ and AUC_{0-∞} to a 400 mg raltegravir dosage form containing crystalline raltegravir.

14. A method of reducing adverse effects upon administration of raltegravir, comprising
administering to the human subject a solid oral dosage form comprising 300 mg of amorphous raltegravir or an equivalent amount of a pharmaceutically acceptable salt thereof.

15. The method of claim 14, wherein the subject is an adult, a child 12 years of age or older, or a child 6 to less than 12 years of age.

16. The method of claim 14, wherein the inter subject coefficient of variability for Cmaxis less than 60%.

17. The method of claim 14, wherein the solid oral dosage form provides only one peak concentration when blood concentrations are plotted against time after administration.

18. The method of claim 14, wherein administering is twice per day.

19. The method of claim 14, wherein the solid oral dosage form comprises a polymer selected from polyethylene oxide and polymethacrylate polymers.

20. The method of claim 14, wherein the 300 mg raltegravir dosage form is bioequivalent in Cmax, AUC₀₋ₜ and AUC_{0-∞} to a 400 mg raltegravir dosage form containing crystalline raltegravir.
